(19) United States
(12) Patent Application Publication
(10) Pub. No.: US 2013/0260117 A1
(43) Pub. Date: Oct. 3, 2013

(54) FLEXIBLE SOFT-FEELING IDENTIFICATION DEVICE

(71) Applicant: ZIH CORP., Hamilton (BM)

(72) Inventor: Gene A. Hofer, Lake Zurich, IL (US)

(73) Assignee: ZIH CORP., Hamilton (BM)

(21) Appl. No.: 13/729,531

(22) Filed: Dec. 28, 2012

Related U.S. Application Data
(60) Provisional application No. 61/616,217, filed on Mar. 27, 2012.

(51) Int. Cl. A61B 5/117

(52) U.S. Cl. CPC A61B 5/117
USPC 428/213; 428/518; 428/523; 428/334; 428/483; 428/424.2; 428/497

(57) ABSTRACT

Identification devices (e.g., wristbands) including a face stock comprising a tough polyolefin material and a flexible vinyl layer are provided. A direct thermal chemistry layer can be deposited directly onto a top surface of the face stock or alternatively a pre-print color layer can be depositing directly onto the top surface of the face stock and the direct thermal chemistry layer can be deposited/located onto the free side of the pre-print layer. The flexible vinyl layer can be adhered to a bottom side of the face stock via an adhesive tie-layer sandwiched between the bottom side of the face stock and the flexible vinyl layer. If desired, an anti-cling layer can also be incorporated into the identification devices.

### CROSS-REFERENCE TO RELATED APPLICATIONS

This application claims the benefit of U.S. Provisional Application No. 61/616,217, filed on Mar. 27, 2012, which is incorporated herein by reference in its entirety.

### TECHNICAL FIELD

The presently disclosed subject matter is related to identification devices (e.g., on demand direct thermal wristbands) that exhibit a desirable level of flexibility and comfort for an individual wearing the device.

### BACKGROUND

Positive patient identification is the foundation of patient safety in the healthcare field. Accordingly, identification devices (e.g., wristbands) must be durable, stay on the patient, and remain readable after repeated use and extended exposures to a variety of environments. Recent approaches for providing a reliable means of patient identification include the use wristbands having bar codes or other identification indicia printed thereon. These devices beneficially avoid the need for handwritten or typed inserts, or stick-on labels, which saves time and eliminates a potential point of failure. Such devices, however, can be irritating to the skin of a wearer of the device. Accordingly, there remains a need for identification devices that provide a desirable level of flexibility and comfort for an individual wearing the device while also maintaining resistance to various environmental conditions that may deteriorate the integrity of the device itself or indicia printed thereon.

### BRIEF SUMMARY

One or more embodiments of the present invention may address one or more of the aforementioned problems. Certain embodiments according to the present invention provide an identification device (e.g., wristband) including a face stock comprising a tough polyolefin material, such as polypropylene, polyester, or polyethylene. The face stock is positioned either directly or indirectly between a flexible vinyl layer and a direct thermal chemistry layer. For instance, the direct thermal chemistry layer can be deposited directly onto a top surface of the face stock or alternatively a pre-print color layer can be depositing directly onto the top surface of the face stock and the direct thermal chemistry layer can be deposited/located onto the free side of the pre-print layer. Preferably, the flexible vinyl layer is directly adhered to a bottom side of the face stock via an adhesive tie-layer sandwiched between the bottom side of the face stock and the flexible vinyl layer.

According to certain embodiments of the present invention, identification devices can comprise a face stock having a top surface and a bottom surface comprising a polyolefin having a low elongation in the machine direction. In this regard, the face stock provides strength and low elongation to prevent stretching or tearing during a printing process or daily use after having indicia printed thereon. A surface, preferably the bottom surface, of the face stock can be laminated to a flexible vinyl layer. For example, an adhesive tie-layer can be positioned between the bottom surface of the face stock and a top surface of a vinyl flexible layer to adhere the two layers together to provide a single element or core to which additional layers above and/or below can be located. In certain embodiments, for instance, a UV curable anti-cling coating can be positioned underneath and at least one of proximate or adjacent to at least a portion of the flexible vinyl layer. In this regard, the flexible vinyl layer can be either directly or indirectly sandwiched between the face stock and the anti-cling coating. Preferably, the anti-cling coating is provided as the final layer of the wristband on the side intended to be in contact with a wearer's skin (i.e., the innermost layer of the device when worn by an individual). Such devices can also comprise a direct thermal chemistry layer positioned above and at least one of proximate or adjacent to at least a portion of the top surface of the face stock. If desired, a pre-print layer can be disposed over the top of the direct thermal chemistry layer such that the direct thermal chemistry layer is positioned between the top surface of the face stock and pre-print layer. Alternatively, the pre-print layer can be positioned underneath the direct thermal chemistry layer such that the pre-print layer is located between the top surface of the face stock and the direct thermal chemistry layer.

In certain preferred embodiments according to the present invention, a UV curable over-varnish, optionally comprising antimicrobial compounds, can be positioned above and at least one of proximate or adjacent to at least a portion of the direct thermal chemistry layer. Preferably, the UV curable over-varnish is clear to an individual's naked eye and/or light to and from a reading device (e.g., clear to light emitted from a bar code reader and light reflected back to the bar code reader from indicia, or unprinted gaps between indicia marks, imprinted on the identification device). The UV curable over-varnish beneficially helps protect the direct thermal image (in the case that indicia have already been formed via a direct thermal printing process) from various types of image degrading substances. These substances can degrade the formed image by reversing the direct thermal image chemistry thereby causing the image to fade in varying degrees. The UV curable over-varnish also beneficially helps protect the direct thermal chemistry layer prior to being subjected to a direct thermal printing process from substances that can cause the background direct thermal chemistry to pre image (darken). Preferably, the UV curable over-varnish is provided as the outermost layer of the wristband on the side intended to be exposed to the environment (i.e., the side not contacting a wearer's skin).

In accordance with certain embodiments, the identification devices can comprise a face stock and an anti-cling layer. In certain such embodiments, the identification devices can, if desired, be devoid of the combination of the polyolefin-based face stock/flexible vinyl layer. Preferably, the anti-cling layer is positioned below and at least one of proximate or adjacent to the bottom surface of the face stock. In certain embodiments, the anti-cling layer comprises a UV curable material. The anti-cling layer can be positioned adjacent (e.g., in direct contact) to the face stock (e.g., adjacent the bottom surface, top surface, or both surfaces of the face stock) or various intermediate layers can be sandwiched between the face stock and the anti-cling layer. For instance, an adhesive intermediate layer (e.g., an adhesive tie-layer) can be sandwiched between the face stock (e.g., the bottom surface of the face stock) and the anti-cling layer. In certain preferred embodiments, the identification devices further comprise a direct thermal chemistry layer positioned above and at least one of proximate of adjacent to at least a portion of the top surface of the face stock. Most preferably, the anti-cling layer comprises the outermost layer on at least the side of the device contacting a user's skin. In this regard, a user (or inanimate object) utilizing the identification devices will enjoy the benefits of having an anti-cling layer in contact with their skin.

In one aspect, the identification device can comprise a medical device provided in the form of a wristband and the indicia are identifying indicia for a patient. In another aspect, the identification device is a flexible wristband that is generally rectangular and includes a pair of opposing long edges and a pair of opposing short edges. The face stock includes a body portion that supports the other layers (flexible vinyl layer, direct thermal chemistry layer, etc.) and any indicia printed thereon and a connector portion that is configured to be secured at its free end to the body portion when encircling a wrist or appendage.

Connectors may be used to connect the connector portion to the body portion, such as a clasp or clip that uses an insert for passing through communicating holes in the body and connector portions. Alternatively, a crack and peel or adhesive/liner connector may be employed that includes an adhesive patch covered by a cover/liner. In certain embodiments according to the present invention, removal of the cover or liner reveals the adhesive patch which can be pressure sensitive and adheres the opposing ends of the face stock together when the ends are applied to form the loop of the wristband.

Embodiments of the present invention provide many advantages. For example, identification devices (such as a wristband) of the present invention can have indicia printed thereon and be used within potentially contaminated environments that require on-demand printing with variable information, such as different forms of identification information. For example, in a hospital environment, patients may be provided identification wristbands that include indicia information such as name, sex, and medical history.

### BRIEF DESCRIPTION OF THE SEVERAL VIEWS OF THE DRAWING(S)

Having thus described embodiments of the invention in general terms, reference will now be made to the accompanying drawings, which are not necessarily to scale, and wherein:

FIG. 1 is a plain view of an identification wristband of one embodiment of the present invention including a crack and peel fastener;

FIG. 2 is a plain view of an identification wristband according to another embodiment of the present invention including a crack and peel fastener;

FIG. 3 is a plain view of an identification wristband according to yet another embodiment of the present invention including a clip fastener;

FIG. 4 is a plain view of an identification wristband according to still another embodiment of the present invention including a clip fastener;

FIG. 5 illustrates a master roll of material containing identification devices being conveyed in the machine direction past a printhead;

FIG. 6 is a cross-sectional diagram of an identification wristband including a polyolefin face stock laminated to a flexible vinyl layer;

FIG. 7 is a cross-sectional diagram of an identification wristband including an anti-cling layer;

FIG. 8 is a cross-sectional diagram of one preferred identification wristband; and

FIG. 9 is a flow diagram for methods of fabricating identification wristbands according to certain embodiments of the present invention.

### DETAILED DESCRIPTION

The present invention now will be described more fully hereinafter with reference to the accompanying drawings, in which some, but not all embodiments of the invention are shown. Indeed, this invention can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

In general, the present invention is, at least in one respect, directed to on demand printed direct thermal identification devices including both a tough face stock having a low elongation percentage in the machine direction to provide a prerequisite strength and a flexible vinyl layer to provide the identification device additional flexibility. Embodiments in accordance with the present invention provide identification devices, such as wristbands, with improved flexibility, pliability, and comfort relative to corresponding devices that art devoid of the combination of the tough face stock and flexible vinyl layer.

As used herein, the term “identification device” may also apply to any devices benefiting from, or requiring, on demand direct thermal printing. For example, within the purview of the present invention are plaques, plates, wristbands, and identification badges bearing (or requiring) indicia printed thereon. Notably, the identification device could be a component of an overall larger device, such as an implant, bandage, handcuffs, or personalized article of clothing if so desired.

The term “face stock” as used herein denotes generally the type of material used to form the supporting structure of the wristband or other identification device and is capable of retaining printed indicia. The term “antimicrobial” as used herein denotes a substance with the ability to control the breeding, growing and proliferation of microorganisms.

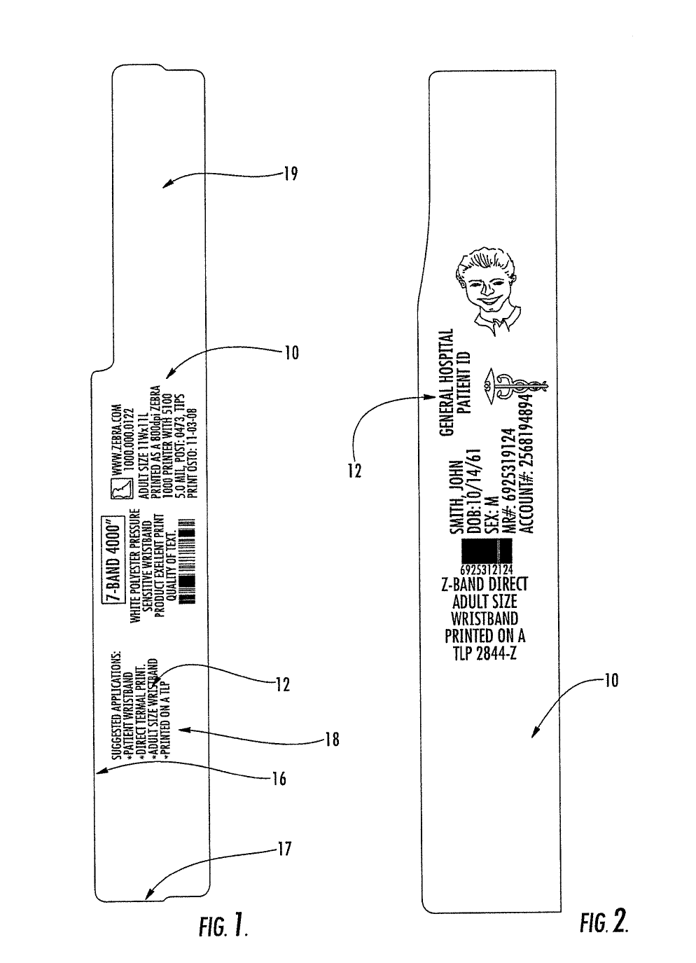

Referring to FIG. 1, identification devices (e.g., wristbands) 10 according to embodiments of the present invention can include a pair of long edges 16, a pair of short edges 17, a body portion 18 and a connector portion 19. The body portion 18 can be generally rectangular and defined between about two-thirds of the two long edges 16 and one of the short edges 17. In certain embodiments, the body portion 18 can be relatively wider than the connector portion 19. The connector portion 19 can also be generally rectangular if desired, but typically (although not in all embodiments) is narrower and shorter than the body portion 18, being defined between the remaining one third of the two long edges 16 and the other one of the short edges 17.

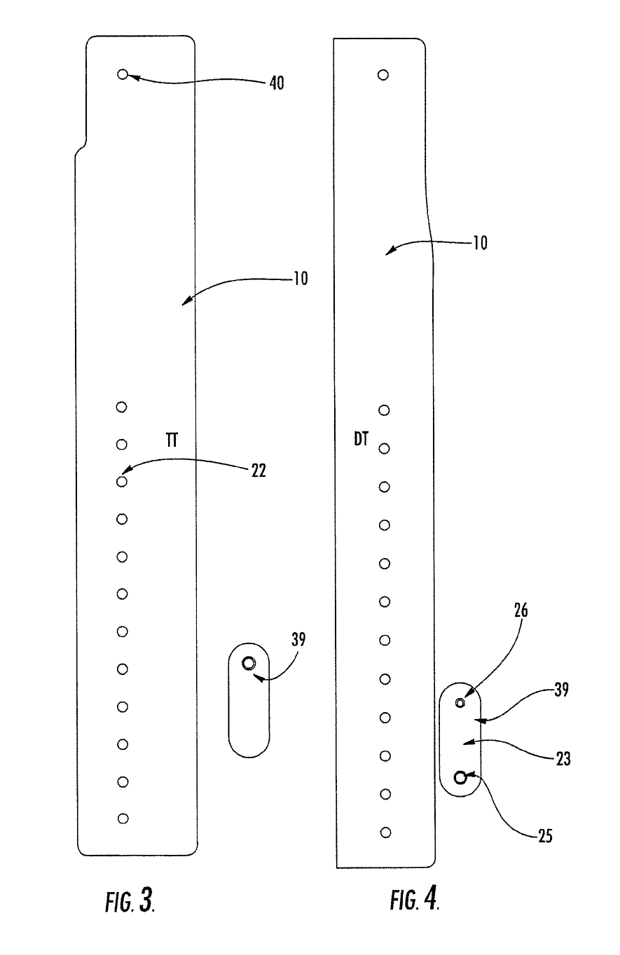

Having the body portion 18 being relatively wide provides a good supporting surface for the indicia 12, which is illustrated in the embodiments shown in FIGS. 1 and 2. FIG. 1 illustrates an embodiment including three panels of print indicia and a barcode symbol. The connector portion 19 is relatively slender, allowing it to be wrapped around a wrist or appendage of a wearer first and then covered over by the body portion 18 so that the indicia 12 remain visible.

In the embodiment illustrated in FIG. 1, the wristband 10 is attached to the user through the use of an adhesive patch (not shown) supported on an underside of the body portion 18 near the short edge or on the topside of the connector portion 19 near the short edge (or both), as shown in FIG. 1. A cover (not shown) extends over the adhesive patch and can be removed using a “crack and peel” method. In this method, the user exposes the adhesive patch located on one end of the wristband and wraps the adhesive end onto the opposite end to secure the wristband in a closed loop. The cover, for example, may include a polyester release liner and the adhesive patch an acrylic adhesive that is pressure sensitive, allowing it to be compressed between the body and connector portions 18, 19 for sealing.

Other fasteners can be used to connect the wristband 10 according to embodiments of the present invention into a loop. For example, in the embodiments illustrated in FIGS. 3 and 4, the wristband 10 includes a clip 39 (e.g., plastic clip, metal clip, paper-based clip, etc. . . . ) which connects to the opposing end via one of a plurality of holes 22. In particular, one of the holes 40 is positioned near the free end of the connector portion 19 and a line of the holes 22 are spaced along the body portion 18. The clip 39 includes a base which is a plastic rectangular sheet with rounded ends. Across a middle portion of the base is a thinned section 24, at one end is supported an opening 25 and at the other end is supported an insert 26. The thinned section 24 allows the base to be bent over on itself for insertion of the insert 26 through an overlapping pair of the holes 22, 40 (when the portions 19, 20 are overlapped to form the loop) and into the opening 25, thereby locking the ends of the wrist band 10 together. Advantageously, the plurality of holes allows the user to adjust the diameter of the band to fit their wrist.

Dimensionally, the wristband 10 of the present invention can vary in overall length, width and thicknesses, although they typically could be sized to fit most persons. For example, the wristband may be 1 inch wide, 11 inches long and about an eighth of an inch thick and fit most normal adult persons. Wristbands intended to be used by infants or small children may be shorter than those intended to be worn by adults. Of course the dimensions and shapes of the identification devices can be varied for attachment to animals, plants, or other objects.

In certain embodiments, the thickness of the entire identification device can comprise a caliper rating in an amount of at least any of the following: 3, 4, and 5 mils; and/or at most about any of the following: 12, 11, 10, 9, 8, 7, and 6 mils. For instance, certain embodiments comprise a caliper of from 4 to 8 mils.

The body portion of the wristband 10 can have other shapes and lengths and still be within the purview of the present invention. For example, the body portion 18 may be three fourths of the length of the wrist band 10 and transition therefrom to the connector portion 19 by an inward slope along one of the long edges 16, as shown in FIG. 2. This differs from the sudden notch that marks the transition between the body portion 18 and connector portion 19 on the embodiment shown in FIG. 1. FIGS. 3 and 4 illustrate still other embodiments of the wristband 10 of the present invention with variations in overall shape. In other embodiments, identification devices such as the wristbands shown in FIGS. 1-4 can be further configured to include one or more contact and/or non-contact systems able to receive and/or transmit data, including but not limited to radio frequency identification (RFID) functionality, near field communication (NFC) functionality, ultra-high frequency functionality (UHF), high frequency (HF) functionality, and Gen2 functionality.

Generally speaking, identification devices (e.g., wristbands) according to embodiments of the present invention can include a face stock comprising a tough polyolefin material and a flexible vinyl layer. A direct thermal chemistry layer can be deposited directly onto a top surface of the face stock or alternatively a pre-print color layer can be depositing directly onto the top surface of the face stock and the direct thermal chemistry layer can be deposited/located onto the free side of the pre-print layer. The flexible vinyl layer can be adhered to a bottom side of the face stock via an adhesive tie-layer sandwiched between the bottom side of the face stock and the flexible vinyl layer. If desired, an anti-cling layer can also be incorporated into the identification devices.

In certain embodiments according to the present invention, however, identification devices identification devices can comprise a face stock and an anti-cling layer. In certain such embodiments, the identification devices can, if desired, be devoid of the combination of the polyolefin-based face stock/flexible vinyl layer. Preferably, the anti-cling layer is positioned below and at least one of proximate or adjacent to the bottom surface of the face stock. In certain embodiments, the anti-cling layer comprises a UV curable material. The anti-cling layer can be positioned adjacent (e.g., in direct contact) to the face stock (e.g., adjacent the bottom surface, top surface, or both surfaces of the face stock) or various intermediate layers can be sandwiched between the face stock and the anti-cling layer. For instance, an adhesive intermediate layer (e.g., an adhesive tie-layer) can be sandwiched between the face stock (e.g., the bottom surface of the face stock) and the anti-cling layer. In certain preferred embodiments, the identification devices further comprise a direct thermal chemistry layer positioned above and at least one of proximate of adjacent to at least a portion of the top surface of the face stock. Most preferably, the anti-cling layer comprises the outermost layer on at least the side of the device contacting a user's skin. In this regard, a user (or inanimate object) utilizing the identification devices will enjoy the benefits of having an anti-cling layer in contact with their skin

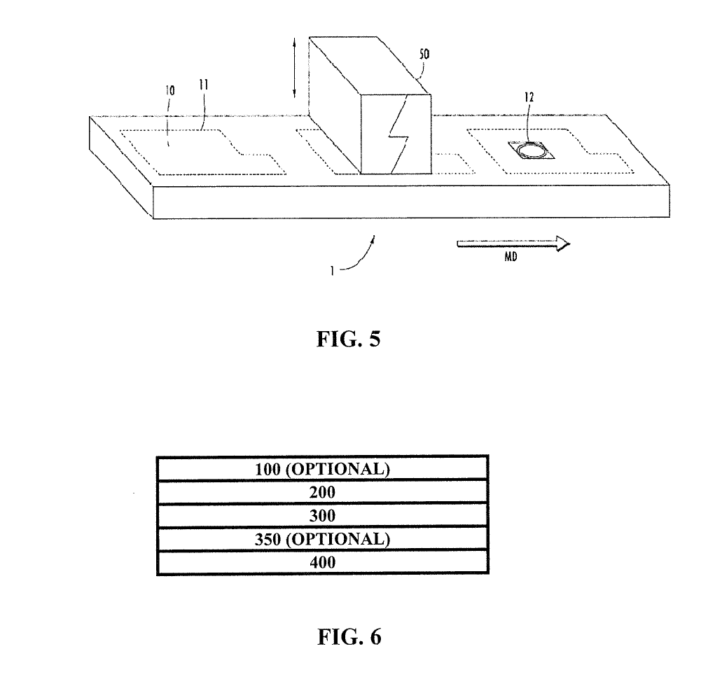

The face stock, in accordance with embodiments of the present invention, can be constructed from a tough and/or stable polyolefin, such as from polypropylene, polyester, or polyethylene. Generally, it is desirable for the face stock to be relatively tough to resist breakage and sufficiently flexible to allow bending into a loop and be able to withstand the heat of printing. However, excessive stretching can cause issues with pre-print registration and make it very difficult to wind rolls that are not extremely tight. The face stock, therefore, can be comprised of a polyolefin having a low elongation percentage in the machine direction. The term “machine direction” as used herein denotes generally the direction in which the identification devices pass or conveyed pass a printhead. As shown in FIG. 5, for example, a master roll 1 of material can comprise multiple individual identification devices 10 removable contained therein by perforations 11 defining the overall shape of the identification devices. The master roll 1 of material can be passed or mechanically conveyed (e.g., pulled) past a printhead 50 in the machine direction as illustrated by the arrow labeled “MD” in FIG. 5. The printhead 50 can raise and lower to engage and disengage the master roll 1 of material as needed to impart indicia 12 on respective identification devices 10. In certain embodiments, “elongation” can be the percent change in length of the material under stress from start to break. However, in certain embodiments, “elongation” can be measured from start to a yield point. In certain preferred embodiments, the elongation percentage is measured according to ASTM D-882 Method B (2009). The face stock, for example, can have an elongation percentage in the machine direction, according to ASTM D-882 Method B (2009), comprising a percentage of at least any of the following: 0.1%, 0.5%, 1%, 2%, 3%, 4%, 5%, and 10%; and/or at most about any of the following: 25%, 20%, 15%, 12%, 10, 5%, 4%, 3%, 2%, 1%, and 0.5%.

In certain embodiments, the face stock can comprise a caliper rating in an amount of at least any of the following: 1, 2, and 3 mils; and/or at most about any of the following: 6, 5, and 4 mils. For instance, in certain embodiments the flexible vinyl layer can comprise a caliper of from 2 to 4 mils.

In certain embodiments of the present invention, the identification devices include a polyolefin-based face stock (e.g., polypropylene, polyester, polyethylene, or combinations thereof). In certain preferred embodiments, the face stock comprises polypropylene. Suitable polyolefin materials for the face stock are commercially available. For instance, an example of one suitable polypropylene material suitable for embodiments of the present invention comprises Ricoh 140LDS polypropylene from Ricoh Electronics (Santa Ana, Calif.). Ricoh 140LDS polypropylene (and the like) provides a durable material being resistant to tearing and stretching or elongation.

The face stock includes a first surface (e.g., a top surface) and a second surface (e.g., a bottom surface). A direct thermal chemistry layer can be positioned above and at least one of proximate or adjacent to at least a portion of the top surface of the face stock. The direct thermal chemistry layer, for example, can be located directly onto the top surface of the face stock. In other words, the direct thermal chemistry layer can be positioned directly adjacent to the top surface of the face stock. Alternatively, a pre-print color layer can be deposited directly onto the top surface of the face stock and the direct thermal chemistry layer can be deposited/located onto the free side of the pre-print layer.

Direct thermal chemistry layers in accordance with certain embodiments of the present invention utilize one or more leuco dyes. Leuco dyes, developers, and sensitizers can be formulated to produce images when appropriate heat and pressure are applied from a direct thermal printer. Resulting images may be black or a variety of colors depending on the leuco dye chemistry. Formulation of leuco dyes, developers, and sensitizers to provide a given color is within the skill of one skilled in the art. In certain embodiments, the direct thermal chemistry layer can be applied, such as to the polyolefin face stock, on press in a liquid state. Preferably, this chemistry is water-based and is dried on press. Particularly desirable results using one or more leuco dye-based formulations can be realized when applied with anilox rolls from 6.0 BCM to 12.0 BCM.

In certain embodiments, the direct thermal chemistry layer can also have a top coat, preferably clear to an individual's naked eye and/or light to and from a reading device (e.g., clear to light emitted from a bar code reader and light reflected back to the bar code reader from indicia, or unprinted gaps between indicia marks, imprinted on the identification device), that provides some level of environmental resistance from substances like: water, isopropyl alcohol, ethyl rubbing alcohol, Purell, hand sanitizers, and sun screen sprays.

A pre-print color layer can comprise any number of colors that may or may not be printed on the identification device. For example, water based flexo inks and/or UV flexo inks can be used to provide a pre-print color layer. Pre-prints can include full color tints or flood coats, spot colors, logos/graphics, alpha numerics and four (4) color process printing. In certain embodiments, the identification devices do not have any color and are only white, clear, or the color of the base material. Desirable performance of devices according to certain embodiments, particularly if used in conjunction with an UV over-varnish, can be realized with a variety of direct thermal printers when the pre-print layer has been deposited using a range of anilox rolls from 3.0 BCM to 6.0 BCM.

The second surface (e.g., a bottom surface) of the face stock is coupled, directly or indirectly, with at least one flexible vinyl layer. The flexible vinyl layer can be positioned below and proximate to the bottom surface of the face stock. In certain embodiments, an adhesive tie-layer is sandwiched between the face stock and the flexible vinyl layer for laminating the two layers together as a unified material or core to which additional layers or coatings can be applied.

The specific vinyl material comprising the flexible vinyl layer is not particularly limited so long as the flexible vinyl layer is capable of being easily bent or flexed. That is, a vinyl material should preferably be selected that is pliable. In other words, the flexible vinyl layer is capable of being easily bent repeatedly without breaking or damaging the layer. The degree of flexibility or stiffness can be ascertained by testing on a Thwing-Albert Handle-O-Meter (e.g., model 3211-55). A Handle-O-Meter generally provides a quantitative value for the stiffness (S) of a material such as for thin polymeric films. The flexible vinyl layer, for example, can have a handle reading provided from a Thwing-Albert Handle-O-Meter (e.g., model 3211-55) comprising at least any of the following: 6, 7, 8, and 9; and/or at most about any of the following: 14, 13, 12, and 11.

The addition of the flexible vinyl layer beneficially provides an improved level of comfort to a wearer. In certain preferred embodiments, the flexible vinyl layer comprises a flexible polyvinyl chloride (PVC). Suitable PVC materials are commercially available, including CONFORMcal™ V 400 SW NO PS from FLEXcon (Spencer, Mass.). In certain embodiments, the flexible vinyl layer can comprise a caliper rating in an amount of at least any of the following: 1, 2, and 3 mils; and/or at most about any of the following: 6, 5, and 4 mils. For instance, in certain embodiments the flexible vinyl layer can comprise a caliper of from 3 to 4 mils.

In certain embodiments, the adhesive tie-layer comprises a pressure sensitive adhesive that is used to laminate the face stock (e.g., polypropylene material) to the flexible vinyl layer (e.g. PVC). Suitable adhesive materials can comprise acrylic or rubber based adhesives. The adhesive tie-layer can comprise a caliper range, for example, from about 0.07 to 1.2 mils. The adhesive material chosen should, preferably, create a secure bond between the face stock (e.g., polypropylene or other material compatible with leuco dye direct thermal chemistry and low elongation in the machine direction). Preferably, the adhesive tie-layer is also capable of surviving exposure to water, sweat, lotions, activity, etc. so that the bond between the connected layers (e.g., face stock and flexible vinyl layer) remains secure. Suitable adhesive are commercially available including Ricoh 140LDS/G25/40# from Ricoh Electronics (Santa Ana, Calif.).

As noted above, the combination of the polyolefin face stock and the flexible vinyl layer beneficially exhibit both strength/durability and flexibility to provide identification devices that exhibit excellent comfort to a wearer. The face stock (e.g., polypropylene layer) provides a stable material with relatively low machine direction elongation properties to laminate on top of the flexible vinyl layer. This construction beneficially enables the identification devices according to certain embodiments of the present invention to be run through converting presses without excessive stretch during the converting process, while also providing an increased level of flexibility and comfort.

Since vinyl films are not compatible with leuco dye direct thermal chemistry, the face stock (e.g., polypropylene) is also needed to provide a compatible surface to retain or support the direct thermal chemistry layer. For instance, coating of leuco dye chemistry directly onto a vinyl base film does not provide acceptable direct thermal performance characteristics for identification device (e.g., wristband) application. One construction that can be employed, therefore, is to laminate the face stock (e.g., polypropylene) to the flexible vinyl layer (e.g., PVC).

The respective caliper ratings (mils) for the face stock and the flexible vinyl layer have already been described. By varying the respective caliper ratings of each layer, a variety of identification devices characterized, in part, by the ratio of the face stock caliper to the flexible vinyl layer caliper provide particularly superior results. In certain embodiments, for example only, the ratio of the face stock caliper to the flexible vinyl layer caliper ranges from about 2:4 to about 4:3, or from about 2:4 to about 4:4, or from about 2:4 to about 2:3.

In certain embodiments, the identification device can also include a UV curable over-varnish positioned above and at least one of proximate or adjacent to at least a portion of the direct thermal chemistry layer. Preferably, the UV curable over-varnish has been applied with anilox rolls in the volume range from about 3 to about 8.5 BCM.

In certain preferred embodiments, the identification devices include a UV over-varnish to help protect the direct thermal image from various types of image degrading substances, such as water, isopropyl alcohol, ethyl rubbing alcohol, Purell, hand sanitizers, sun screen sprays, and soaps. These substances can degrade the image by reversing the direct thermal image chemistry (causing the image to fade in varying degrees) or causing the background direct thermal chemistry to pre image (darken) or both. Preferably, the UV curable over-varnish is provided as the outermost layer of the wristband on the side intended to be exposed to the environment (i.e., the side not contacting a wearer's skin). In one aspect, the medical device is a wristband and the indicia are identifying indicia for a patient.

In certain preferred embodiments, the UV curable over-varnish is clear to an individual's naked eye and/or light to and from a reading device (e.g., clear to light emitted from a bar code reader and light reflected back to the bar code reader from indicia, or unprinted gaps between indicia marks, imprinted on the identification device). For instance, once the over-varnish is cured by a UV light source it protects the direct thermal chemistry layer. In certain embodiments, the indicia imprinted on the identification device (via heat provided to the thermal chemistry layer) can comprise a barcode. To viably read the imprinted barcode, a reading device (e.g., human eye or reading device) should be able to read the indicia (e.g., a barcode) through the over-varnish. Barcode readers, for example, use light (e.g., visible light or IR light) to read imprinted barcodes. Preferably the over-varnish according to certain embodiments of the present invention is clear to such light. That is, the over-varnish allows the light from the barcode reader to pass through the over-varnish, with some of the light hitting a barcode imprinted on the direct thermal chemistry layer. Some of the light passed through the over-varnish is absorbed, while some is reflected back. The light reflected back should preferably be allowed to pass back through the over-varnish to the reading device. The reading device (e.g., bar code reader) can then process what has been reflected back to decode the indicia (e.g., barcode). In certain embodiments, therefore, the over-varnish can be clear to the light emitted from a barcode reader and light coming back from the barcode. The light to and from the indicia (e.g., barcode) is usually, but not necessarily, at the same frequency. It should be noted that neither of these lights need to be at frequencies visible to humans. Accordingly, the over-varnish does not have to look clear to a juror to be clear to a scanner, and it doesn't have to be clear before it is cured. Preferably, the over-varnish is clear to certain frequencies of light, based on the specific application in which it will be used.

The over-varnish can be applied on press during the manufacture of the devices, and preferably utilizes a free radical curing mechanism and is cured on press with UV lamps. That is, light from UV lamps can initiate a free radical polymerization that cures the over-varnish. One example of a UV over-varnish that can be used is iimak F10UVTC015G UV varnish from iimak (Amherst, N.Y.). Coat weight can be selected per desired specific UV formula and performance characteristics including direct thermal image protection and cartridge printer performance (e.g., Zebra HC 100 cartridge printer performance).

Other features may also be added to the over-varnish including antimicrobial compounds (e.g., silver zeolites and triclosans) and covert machine readable counterfeiting/security features. These optional components can be added to the varnish by % weight typically in the 3%-8% range and do not negatively affect the performance of the over-varnish in terms of image protection or ease of use with printers.

Certain identification devices according to embodiments of the present invention include an anti-cling coating positioned underneath and at least one of proximate or adjacent to at least a portion of the flexible vinyl layer. Preferably, the anti-cling coating comprises a UV curable material. In this regard, the flexible vinyl layer can be either directly or indirectly sandwiched between the face stock and the anti-cling coating. Preferably, the anti-cling coating is provided as the final layer of the wristband on the side intended to be in contact with a wearer's skin (i.e., the innermost layer of the device when worn by an individual).

In certain embodiments, the anti-cling layer provides yet further enhancement of the level of comfort of the identification device (e.g., band) for the user. The UV curable anti-cling layer can be applied as a coating to the back/bottom of the flexible vinyl layer. Addition of the anti-cling layer mitigates the tendency of the device to cling to the skin of a wearer. Some flexible vinyl materials, for instance, exhibit a tendency to cling to a wearer's skin upon getting wet. The anti-cling layer mitigates this possibility. Suitable results can be realized when the anti-cling chemistry is applied with anilox rolls from, for example, about 4.0 BCM to about 8.5 BCM.

In certain embodiments, the UV curable anti-cling material comprises at least one acrylated oligomer. Preferably, the acrylated oligomers comprise at least one acrylated organic oligomer. One example of a suitable material for formation of the anti-cling layer is CHUV-8489 UV Soft Feel Matte from Wikoff Color Corp. (Wauconda, Ill.).

In other embodiments, the UV curable material comprises one or more polyols, polyesters, linear hydroxyl-bearing polyesters, polyurethanes, polyester-polyurethane dispersions, anionic polyurethane dispersions. In certain embodiments, the the UV curable material comprises a hydroxyl-containing polyester-urethane.

In certain embodiments, the anti-cling layer has been provided with a modified texture (increased coefficient of friction than realized upon simply depositing the material into position) such that difference in coefficients of friction between the outermost layer of the device (e.g., the over-varnish as the layer exposed to environmental conditions and not adjacent to a wearer's skin) and the inner most body layer (e.g., the anti-cling layer) is realized. A difference in the coefficient of friction between the two outermost layers (e.g., one layer adjacent a wearer's skin and the other facing away from the wearer's skin) of the device beneficially help process the devices through various on demand direct thermal printers. In certain embodiments, the difference of the coefficients of friction between the two outermost layers can comprise a difference of at least any of the following: 1, 3, 5, 10, and 15%; and/or at most about any of the following: 5, 10, 15, 18, 20, 30, 40, and 50%.

FIG. 6 illustrates a cross-sectional diagram of an identification wristband including a polyolefin (e.g., polypropylene) face stock 300 laminated via an adhesive tie-layer 350 to a flexible vinyl layer (e.g., PVC) 400. In this particular embodiment illustrated by FIG. 6, a direct thermal chemistry layer 200 (e.g., formulation comprising one or more leuco dyes) is positioned adjacent the top side of the face stock 300 and a clear UV curable over-varnish 100 is provided as the outermost layer of the wristband (i.e., the side intended to be exposed to the environment, not the side contacting a wearer's skin). As shown in FIG. 6, the particular embodiment illustrated includes optional layers 100 and 350.

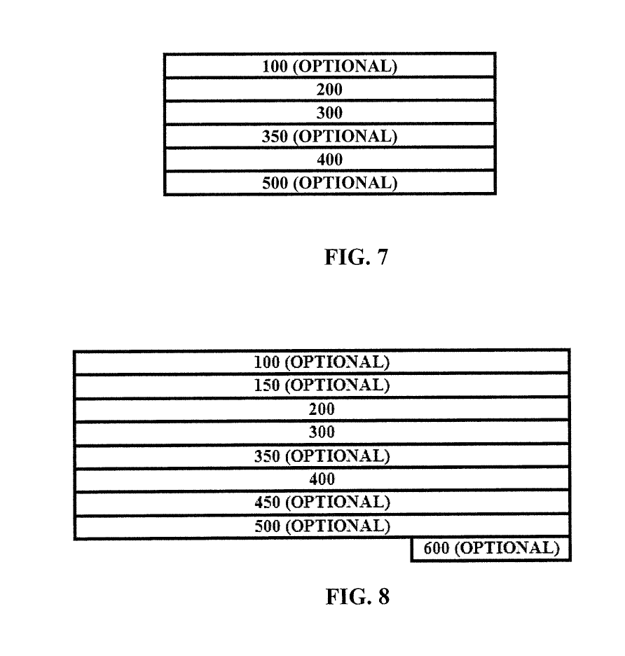

FIG. 7 illustrates a cross-sectional diagram of an identification wristband according to a different embodiment. The embodiment illustrated in FIG. 7 includes a UV curable anti-cling coating 500. As shown in FIG. 7, the UV curable anti-cling coating 500 can be positioned beneath and adjacent to at least a portion of the flexible vinyl layer 400. In this particular embodiment, the anti-cling coating 500 is provided as the final layer of the wristband on the side intended to be in contact with a wearer's skin (i.e., the innermost layer of the device when worn by an individual). As shown in FIG. 7, the particular embodiment illustrated includes optional layers 100, 350, and 500.

FIG. 8 illustrates a cross-sectional diagram of one preferred identification wristband. In addition to the layers present in the embodiment illustrated in FIG. 8, the embodiment illustrated in FIG. 8 includes a pre-print layer 150 sandwiched between the direct thermal chemistry layer 200 and the UV curable over-varnish 100. The embodiment shown in FIG. 8 also includes a registration mark layer 450. The registration mark layer is positioned below the flexible vinyl layer 400 and above the anti-cling coating 500. Preferably, a black UV ink is used to print a printer registration mark on the back (bottom or underside) of the flexible vinyl layer 400. Through testing it was determined that water based black inks did not dry appropriately on the vinyl and caused pick off when the product was wound into rolls. This produced unwanted black marks on the face of the band.

The embodiment shown in FIG. 8 also includes an optional adhesive closure tab 600 that secures the band to itself when applied to the user (or any object that it will fit around). The adhesive closure tab 600 can be applied during the manufacture of the bands and offers a rubber based or acrylic pressure sensitive adhesive. Preferably, the adhesive closure tab 600 is found mostly or completely on the connector portion of the wristband. As illustrated in FIG. 8, for instance, the adhesive closure tab preferably does not significantly overlie the layer comprising the anti-cling coating 500. In such embodiments, therefore, the anti-cling coating 500 can remain the final layer of the body portion of the wristband on the side intended to be in contact with a wearer's skin (i.e., the innermost layer of the device when worn by an individual). In this regard, the adhesive closure tab 600 does not interfere or block contact of the anti-cling coating 500 with a wearer's skin such that the benefits associated with the anti-cling coating are retained. Although the particular embodiment illustrated in FIG. 8 shows only a single adhesive closure tab 600, certain embodiments according to the present invention can include an additional adhesive closure tab on the opposing end of the device if so desired. As shown in FIG. 8, the particular embodiment illustrated includes optional layers 100, 150, 350, 450, 500 and optional adhesive closure tab 600.

The adhesive should secure the band and not easily release. Furthermore, it should offer resistance to water exposure and also should be strong enough to void out tamper evident security slits if the user tires to remove and re-apply the band. A suitable material, for example, includes Fasson 40#SCK/S246/40#SCK, Spec 98819 which is a super-calendered kraft paper used as a strip/throw-away liner for transfer tape applications and utilizes a general purpose permanent rubber based adhesive featuring high initial tack and ultimate adhesion.

In certain embodiments, the combination of the face stock (e.g., polypropylene) and the flexible vinyl layer provides a better material for tamper evident void performance than vinyl alone. The vinyl material alone can be susceptible to merely stretching instead of tearing or voiding out a tamper evident pattern employed by identification device. However, the combination of the face stock (e.g., polypropylene) and the flexible vinyl layer, according to certain embodiments, provides strength and the properties required to void out (e.g., void out a tamper evident pattern) if removed (e.g., the identification device is tampered with). Since the flexible vinyl layer alone would stretch too much for optimum voiding performance, the face stock (e.g., polypropylene) provides some rigidity and helps the band void out as desired.

In another aspect, the present invention provides methods of fabricating identification devices including the combination of a generally durable facestock and a flexible vinyl layer. In certain embodiments, the methods can comprise steps of laminating together a pressure sensitive direct thermal polypropylene label stock (e.g., includes the direct thermal chemistry on one side and an adhesive material on the opposite side) and a flexible vinyl film. In certain embodiments, that is, the facestock material can be provided with the direct thermal chemistry layer already deposited onto a top surface of the facestock and an adhesive material/layer provided on the other or bottom side of the facestock. In such embodiments, the adhesive is generally covered with a release liner prior to the laminating step. When ready to laminate the facestock and the flexible vinyl layer together, the release liner can be delaminated to expose the adhesive material. The facestock and flexible vinyl layer can be laminated together by conventional methods in which the adhesive tie-layer is sandwiched between the facestock and the flexible vinyl layer.

In certain embodiments, however, the facestock is not necessarily provided with the direct thermal chemistry layer and/or an adhesive material. In such cases, a suitable direct thermal chemistry layer (e.g., leuco dyes) can be applied directly to a first side (e.g., top surface) of the facestock by conventional methods either before or after lamination to the flexible vinyl layer. In certain embodiments, however, a pre-print color layer can be depositing directly onto the top surface of the face stock and the direct thermal chemistry layer can be deposited/located onto the free side of the pre-print layer. In embodiments in which the facestock is not provided with an adhesive already deposited thereon, a suitable adhesive can be applied to one side (e.g., bottom surface) of the facestock or one side of the flexible vinyl layer prior to laminating the two components together by conventional methods.

In certain preferred embodiments, an optional registration mark layer is positioned below the flexible vinyl layer (e.g., bottom side or free side of the flexible vinyl layer after being laminated to the facestock). Preferably, a black UV ink is used to print a printer registration mark on the back (bottom or underside) of the flexible vinyl layer. In addition to the optional registration mark, a variety of pre-print colors, coatings, and over-varnishes can be applied to the identification device using print stations (e.g., anilox rolls) as referenced previously. In certain embodiments, for instance, a pre-print color layer can be deposited directly over the top surface of the facestock followed by an application of a UV curable over-varnish. Similarly, an anti-cling coating can be deposited (e.g., printed via anilox rolls) onto the identification devices. Preferably, the resulting identification devices comprise a first or top side surface comprising a UV curable over-varnish and a second or bottom side surface (e.g., closest to the skin of a wearer or user) comprising an anti-cling coating.

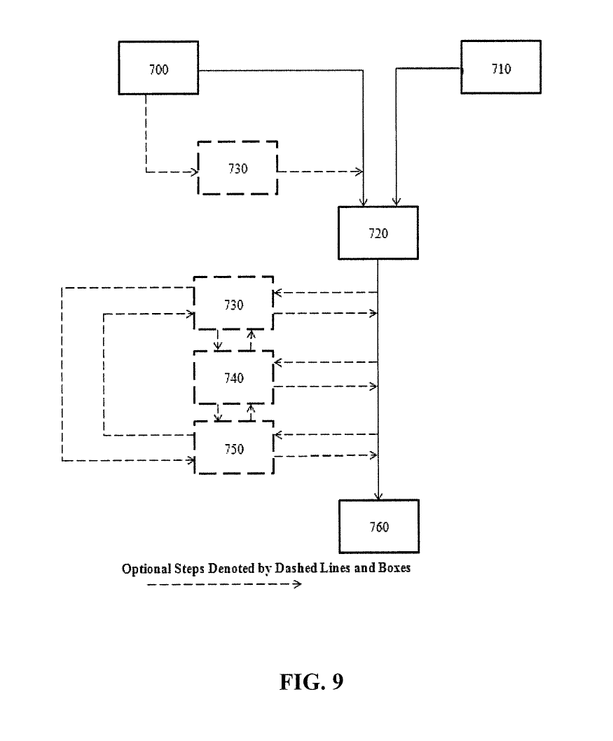

FIG. 9 is a flow diagram for methods of fabricating identification wristbands according to certain embodiments of the present invention. In these particular embodiments, a master roll of a flexible vinyl material 700 and a master roll of pressure sensitive direct thermal polypropylene label stock 710 (e.g., includes the direct thermal chemistry on one side and an adhesive material on the opposite side) are provided. The master roll of pressure sensitive direct thermal polypropylene label stock includes a release liner covering the adhesive material. Although not shown, the release liner from direct thermal polypropylene label stock is delaminated to expose the adhesive material and the master roll of the flexible vinyl material 700 and the master roll of pressure sensitive direct thermal polypropylene label stock 710 are positioned relative to each other such that the adhesive material is positioned between the master roll of the flexible vinyl material 700 and the master roll of pressure sensitive direct thermal polypropylene label stock 710. The facestock plus adhesive is adhered to the flexible vinyl material in a laminating step 720. In embodiments including an optional registration mark layer 450 as illustrated by FIG. 8, the optional registration mark layer can be provided in an optional registration addition step 730 as illustrated by the dashed lines in FIG. 9. That is, the registration addition step 730 can be optionally employed in certain embodiments of the present invention. As illustrated in FIG. 8, for instance, the optional registration addition step 730 can be performed either prior to or after the laminating step 720. In embodiments that employ the optional registration addition step 730, a black UV ink is preferably used to print a printer registration mark on the back (bottom or underside) of the master roll of a flexible vinyl material. FIG. 9 also illustrates an optional coating step 740, as illustrated by the dashed lines on FIG. 9, in which any colors (e.g., pre-print color layers), coatings, and/or varnishes can be applied using print stations. For instance, coating step 740 can actually comprise multiple discrete coating steps in which different layers are added to the identification device. For instance, coating step 740 can comprise a first coating step comprising the application of a pre-print layer substantially overlying the direct thermal chemistry layer provided on the top surface of the facestock, a second coating step comprising the application of a UV curable over-varnish substantially overlying the pre-print color layer, and a third coating step comprising the application of an anti-cling layer to the intended underside of the identification device. In certain embodiments, coating step 740 can include one or more of the above referenced coating steps (e.g., application of pre-print layer, over-varnish, and anti-cling coating). For instance, in certain embodiments coating step 740 comprises a step of applying a UV curable over-varnish and a step of applying an anti-cling coating without including a step of applying a pre-print color layer.

In the embodiments illustrated in FIG. 9, such methods can also include an optional step (as illustrated by dashed lines in FIG. 9) of applying an adhesive tab 750. This step can provide an adhesive closure tab that secures the band to itself when applied to the user (or any object that it will fit around). Preferably, the adhesive tab comprises a rubber based or acrylic pressure sensitive adhesive and is deposited mostly or completely on the connector portion of the wristband. As such, exposure of a wearer's skin to the adhesive tab is mitigated or totally eliminated.

After the individual components of the device (e.g., facestock, flexible vinyl layer, and additional coatings/layers) have been adhered into a unified or integral master roll of material, a die cutting step 760 is preferably performed as illustrated in FIG. 9. The die cutting step 760 comprises cutting the integral master roll of material to create one or more identification devices having predetermined dimensions such as width, length and overall shape (e.g., rounded corners, etc.). In certain embodiments, the die cutting step can provide perforations outlining the overall desired shape of one or more identification devices such that individual identification devices can be removed one at a time when needed. In this aspect, the individual identification devices remain contained within the master roll and can be wound into one or more finished rolls of material including a plurality of identification devices that can be printed onto and/or dispensed and/or removed individually upon demand.

In certain embodiments, the identification devices formed during the die cutting step 760 can be provided in the form of a fanfold, in which adjacent identification devices are stacked over the top of each other and connected by a respective folded portion (e.g., a crease) located between two adjacent identification devices removably contained (e.g., perforations outlining the respective identification devices) within the cut master roll of material. In this regard, such embodiments can provide a web of a cut master roll material folded onto itself, preferably multiple times, to form a fan-shaped stack of identification devices. Preferably, a single identification device is positioned between each respective fold. However, multiple identification devices can be positioned between each respective fold (e.g., 2-10 devices located between respective folds).

In certain other embodiments, however, the die cutting step 760 can form discrete identification devices that are completely separated from the master roll of material. In this regard, the die cutting step can cut-out one or more separate identification devices from the integral master roll of material. In such embodiments, the individual identification devices can be provided individually (e.g., a container including one or more identification devices) or dispensed individually from a cartridge or similar dispenser housing one or more discrete identification devices.

As noted above, FIG. 9 illustrates methods of fabricating identification wristbands according to certain embodiments of the present invention. FIG. 9 shows several optional steps (i.e., 730, 740, and 750) that can be incorporated individually (solely) or in any combination (e.g., 730, 740, and 750; 740 and 750; etc.) as illustrated by the dashed lines in FIG. 9.

Many modifications and other embodiments of the invention set forth herein will come to mind to one skilled in the art to which these inventions pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An identification device, comprising:
(a) a face stock having a top surface and a bottom surface, said face stock comprising a polyolefin;
(b) a flexible vinyl layer positioned below and proximate to the bottom surface of the face stock;
(c) a direct thermal chemistry layer positioned above and at least one of proximate or adjacent to at least a portion of the top surface of the face stock.

2. The device according to claim 1, further comprising an adhesive tie-layer sandwiched between the face stock and the flexible vinyl layer.

3. The device according to claim 1, wherein the direct thermal chemistry layer is positioned directly adjacent to the top surface of the face stock.

4. The device according to claim 3, wherein the direct thermal chemistry layer comprises one or more leuco dyes.

5. The device according to claim 1, wherein the face stock comprises a caliper from about 2 to about 4 mils.

6. The device according to claim 1, wherein the flexible vinyl layer comprises a caliper from about 3 to about 4 mils.

7. The device according to claim 1, wherein the ratio of the face stock caliper to the flexible vinyl layer caliper ranges from about 2:4 to about 4:3.

8. The device according to claim 1, further comprising an anti-cling coating positioned underneath and at least one of proximate or adjacent to at least a portion of the flexible vinyl layer.

9. The device according to claim 8, wherein the anti-cling coating comprises a UV curable material.

10. The device according to claim 9, wherein the UV curable material comprises at least one acrylated oligomer.

11. The device according to claim 9, wherein the UV curable material comprises at least one acrylated organic oligomer.

12. The device according to claim 9, wherein the UV curable material comprises one or more polyols, polyesters, linear hydroxyl-bearing polyesters, polyurethanes, polyester-polyurethane dispersions, anionic polyurethane dispersions.

13. The device according to claim 9, wherein the UV curable material comprises a hydroxyl-containing polyester-urethane.

14. The device according claim 9, wherein the anti-cling coating has been applied with anilox rolls in the volume range from about 4.0 to about 8.5 BCM.

15. The device according claim 1, wherein the face stock comprises polypropylene, polyester, polyethylene, or combinations thereof.

16. The device according to claim 1, wherein the flexible vinyl layer comprises polyvinyl chloride.

17. The device according to claim 1, further comprising a UV curable over-varnish positioned above and at least one of proximate or adjacent to at least a portion of the direct thermal chemistry layer.

18. The device according to claim 17, wherein the UV curable over-varnish has been applied with anilox rolls in the volume range from about 3 to about 8.5 BCM.

19. The device according to claim 17, wherein the over-varnish comprises antimicrobial compounds.

20. An identification device, comprising:
(a) a face stock having a top surface and a bottom surface, said face stock comprising a polyolefin having a low machine direction elongation;
(b) a flexible vinyl layer positioned below and proximate to the bottom surface of the face stock;
(c) an adhesive tie-layer positioned between the face stock and the flexible vinyl layer;
(d) a UV curable anti-cling coating comprising at least one acrylated oligomer, said UV curable anti-cling layer being positioned underneath and at least one of proximate or adjacent to at least a portion of the flexible vinyl layer;
(e) a direct thermal chemistry layer positioned above and at least one of proximate or adjacent to at least a portion of the top surface of the face stock; and
(f) a UV curable over-varnish comprising antimicrobial compounds, said UV curable over-varnish being positioned above and at least one of proximate or adjacent to at least a portion of the direct thermal chemistry layer.

21. An identification device, comprising:
(a) a face stock having a top surface and a bottom surface; and
(b) an anti-cling layer positioned below and at least one of proximate or adjacent to the bottom surface of the face stock.

22. The device according to claim 21, wherein the anti-cling layer comprises a UV curable material.

23. The device according to claim 21, wherein the anti-cling layer is positioned adjacent the bottom surface of the face stock.

24. The device according to claim 21, further comprising an intermediate adhesive tie-layer directly or indirectly sandwiched between the anti-cling layer and the bottom surface of the face stock.

25. The device according to claim 21, further comprising a direct thermal chemistry layer positioned above and at least one of proximate of adjacent to at least a portion of the top surface of the face stock.

26. The device according to claim 21, wherein the anti-cling layer comprises the outermost layer on at least the side of the device contacting a user's skin.
